(19) United States
(12) Patent Application Publication
(10) Pub. No.: US 2025/0032661 A1
(43) Pub. Date: Jan. 30, 2025

(54) HYDROCOLLOID ADHESIVES AND WOUND DRESSINGS CONTAINING A HYDROCOLLOID ADHESIVE

(71) Applicant: DelStar Technologies, Inc, Alpharetta, GA (US)

(72) Inventors: Pranabes PRAMANIK, Alpharetta, GA (US); Sarah AANONSEN, Alpharetta, GA (US); Paula ZIEGLER, Alpharetta, GA (US); Nick SHAFFER, Alpharetta, GA (US)

(21) Appl. No.: 18/775,305

(22) Filed: Jul. 17, 2024

Related U.S. Application Data
(60) Provisional application No. 63/515,881, filed on Jul. 27, 2023.

(51) Int. Cl. A61L 15/58; A61F 13/00; A61F 13/0246

(52) U.S. Cl. CPC A61L 15/585; A61F 13/0253; A61F 2013/00659

(57) ABSTRACT

Hydrocolloid adhesive compositions, wound dressings comprising hydrocolloid adhesive compositions and methods of producing such compositions are provided. A hydrocolloid adhesive composition comprises a base polymer, a hydrocarbon tackifier, an absorbent, and a plasticizer. The hydrocarbon tackifier may comprise a cycloaliphatic hydrocarbon modified with aromatic functional groups. A wound dressing comprising the hydrocolloid layer is disposed on a carrier film. The wound dressing may comprise a discontinuous hydrocolloid layer. The hydrocolloid adhesive compositions are substantially transparent to improve visibility through a dressing. The compositions may further include materials that reduce allergic reactions by the user.

### CROSS-REFERENCE TO RELATED APPLICATIONS

This application claims the benefit of U.S. Provisional Application Ser. No. 63/515,881, filed Jul. 27, 2023, the complete disclosure of which is incorporated herein by reference for all purposes.

### BACKGROUND

The production of wound exudate or fluid from a mammalian wound is important for wound healing. Exudate is an acidic solution that mainly contains water, but may also contain electrolytes, nutrients, proteins, inflammatory mediators, protein digesting enzymes, growth factors and various types of cells (e.g., white blood cells). While wound healing generally benefits from a moist environment, too much exudate may delay healing and detrimentally affect a patient's quality of life.

Wound dressings may be designed to assist in maintaining a moist wound environment and/or managing exudate accumulation in the wound and peri-wound area. For example, wound dressings may be designed to absorb excess wound exudate and/or allow transmission of water vapor through the dressing.

A medical practitioner may be required to check the progress of wound healing and decide when a wound dressing should be changed (e.g., when it is saturated). However, this is often difficult to judge since the wound dressing may need to be removed completely to determine the progress of wound healing.

Many wound dressings contain hydrocolloid adhesives. These wound dressings may comprise a carrier film or backing layer and a hydrocolloid adhesive layer. The hydrocolloid adhesive layer provides a number of functions, including adhering the dressing to the skin and absorbing the wound exudate.

A frequent component of hydrocolloid adhesives is colophony (also known as rosin), which is included in the hydrocolloid adhesive as a tackifier. Colophony is derived from the sap of coniferous trees such as pines, junipers, firs and cedars. Although colophony is a useful plant-based tackifier for use in wound dressings, it is also a known allergen that can cause, for example, contact dermatitis.

### SUMMARY

Adhesive compositions and wound dressings with adhesive compositions are provided. The adhesives include hydrocolloid compositions that are substantially transparent to improve visibility through a dressing. The compositions may further include materials that reduce allergic reactions by the user.

Certain embodiments may provide one or more of the following additional advantages: (1) transparency of the hydrocolloid adhesives, which allows the production of see-through wound dressings that improve observation of wounds through the dressing; (2) reduced incidence of allergic reactions through the use of allergen-free compositions; (3) use of easily available and low cost components to improve efficiency and cost effectiveness; (4) improved compatibility with a range of different carrier film materials, including, for example, ethylene vinyl acetate films; (5) improved cold flow characteristics; (6) improved visibility of wounds through a dressing even after absorption of significant amounts of wound exudate; and (7) increased flexibility of the dressing.

In one aspect, a hydrocolloid adhesive composition comprises a base polymer, a hydrocarbon tackifier, an absorbent and a plasticizer. The hydrocarbon tackifier may comprise a cycloaliphatic hydrocarbon modified with aromatic functional groups.

The hydrocolloid adhesive composition may comprise about 5 wt. % to about 20 wt. % base polymer, about 25 wt. % to about 40 wt. % hydrocarbon tackifier, about 30 to about 50 wt. % absorbent, and/or up to about 20 wt. % plasticizer.

In one embodiment, the hydrocolloid adhesive composition comprises about 10 to about 15 wt. % base polymer, about 25 to about 35 wt. % hydrocarbon tackifier, about 41 to about 46 wt. % absorbent and about 6 to about 17 wt. % plasticizer.

In another embodiment, the hydrocolloid adhesive composition comprises about 10 wt. % to about 15 wt. % poly(styrene-isoprene-styrene), about 25 wt. % to about 35 wt. % hydrocarbon tackifier, about 41 wt. % to about 46 wt. % carboxymethylcellulose, about 5 wt. % to about 10 wt. % mineral oil and about 1 wt. % to about 8 wt. % liquid isoprene rubber.

In various embodiments, the cycloaliphatic hydrocarbon modified with aromatic functional groups comprises about 5 wt. % to about 15 wt. % aromatic protons. The cycloaliphatic hydrocarbon may have a softening point of from about 80° C. to about 100° C.

In various embodiments, the base polymer comprises a poly(styrene-olefin-styrene) polymer or a poly(styrene-isoprene-styrene) polymer.

In various embodiment, the absorbent is a hydrophilic polymer. The hydrophilic polymer may comprise a cellulose.

In various embodiments, the plasticizer is selected from the group consisting of mineral oil, liquid isoprene rubber and combinations thereof.

In another aspect, a hydrocolloid adhesive composition comprises a base polymer, a tackifier, an absorbent and a plasticizer. The hydrocolloid adhesive composition is substantially transparent. “Transparent” as used herein means that the material transmits incident light with negligible scattering and little absorption, enabling a wound to be seen through the transparent wound dressing (or the transparent part of the wound dressing) under typical, unaided viewing conditions.

In various embodiments, the hydrocolloid adhesive composition has a refractive index ranging from about 1 to about 2, or about 1 to about 1.9, or about 1 to about 1.8 or about 1 to about 1.7. The composition may have a total transmittance of at least about 50%, or at least about 55%, or at least about 60%, or at least about 65%, or at least about 70%, or at least about 75%, or at least about 80%, or at least about 85%, or at least about 90%.

In another aspect, a hydrocolloid adhesive composition comprises a base polymer, a hydrocarbon tackifier, an absorbent and a plasticizer. The hydrocarbon tackifier is a cycloaliphatic hydrocarbon modified with aromatic functional groups.

In various embodiments, the cycloaliphatic hydrocarbon modified with aromatic functional groups comprises about 5 wt. % to about 15 wt. % aromatic protons.

In various embodiments, the cycloaliphatic hydrocarbon modified with aromatic functional groups has a softening point of from about 80° C. to about 100° C.

In various embodiments, the cycloaliphatic hydrocarbon modified with aromatic functional groups has a number average molecular weight of about 300 g/mol to about 600 g/mol. The cycloaliphatic hydrocarbon may have a weight average molecular weight of about 600 g/mol to about 800 g/mol. The cycloaliphatic hydrocarbon modified with aromatic functional groups may comprise a C3 to C10 cycloaliphatic hydrocarbon resin.

In another aspect, a wound dressing comprises a carrier film and a hydrocolloid layer disposed on the carrier film. The hydrocolloid layer may comprise a hydrocolloid adhesive composition comprising a base polymer, a hydrocarbon tackifier, an absorbent and a plasticizer.

In various embodiments, the carrier film is selected from the group consisting of a polyurethane film, an ethylene vinyl acetate film, or a combination thereof.

The hydrocolloid layer may be a discontinuous layer of hydrocolloid. The hydrocolloid layer may comprise a plurality of undulating lines, dots or parallel lines disposed on the carrier film.

The hydrocolloid adhesive composition may comprise any hydrocolloid adhesive composition described herein. In some examples, the hydrocolloid adhesive composition may comprise about 5 wt. % to about 20 wt. % base polymer, about 25 wt. % to about 40 wt. % hydrocarbon tackifier, about 30 to about 50 wt. % absorbent, and up to about 20 wt. % plasticizer.

In various embodiments, the wound dressing further comprises an additional adhesive disposed between the carrier film and the hydrocolloid layer such that the carrier film is in contact with the hydrocolloid layer. The additional adhesive may be translucent or transparent.

In various embodiments, the wound dressing has a refractive index ranging from about 1 to about 2, or about 1.0 to about 1.7. The wound dressing may have a total transmittance of at least about 50%, or at least about 90%.

In another aspect, there is provided a method of producing a wound dressing comprising depositing a hydrocolloid adhesive composition on a carrier film to form a hydrocolloid layer. The hydrocolloid adhesive composition may comprise any hydrocolloid adhesive composition described herein. In some examples, the hydrocolloid adhesive composition may comprise about 5 wt. % to about 20 wt. % base polymer, about 25 wt. % to about 40 wt. % hydrocarbon tackifier, about 30 to about 50 wt. % absorbent, and up to about 20 wt. % plasticizer.

The details, examples and preferences provided in relation to any particular one or more of the stated aspects of the present description will be further described herein and apply equally to all aspects of the present description. Any combination of the embodiments, examples, and preferences described herein in all possible variations thereof is encompassed by the present description unless otherwise indicated herein, or otherwise clearly contradicted by context.

### BRIEF DESCRIPTION OF THE DRAWINGS

The accompanying drawings, which are incorporated in and constitute a part of this specification, illustrate an embodiment of the description and, together with the description, serves to explain the principles of the description.

FIG. 1 is a schematic representation of a wound dressing;

FIG. 2 is a schematic representation of another embodiment of a wound dressing;

FIG. 3 is a schematic representation of another embodiment of a wound dressing; and

FIGS. 4A-4C are photographs of different embodiments of wound dressings.

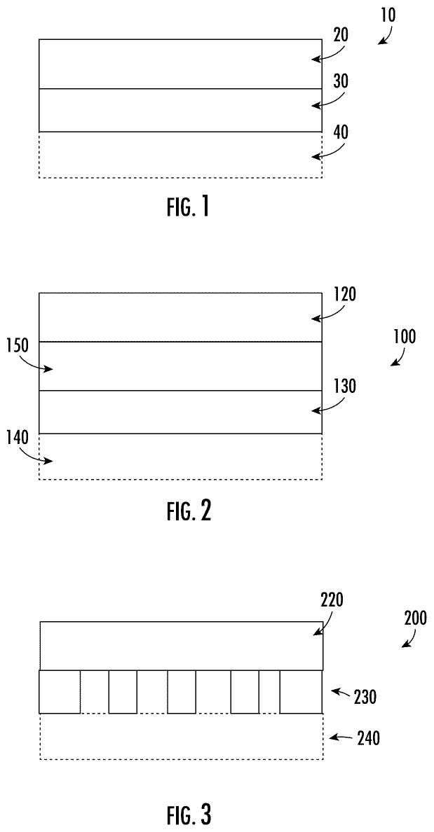

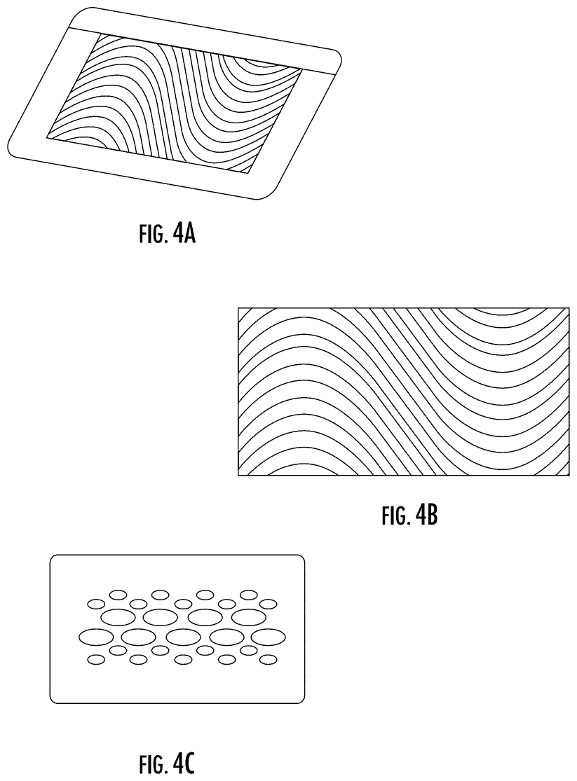

### DETAILED DESCRIPTION

This description and the accompanying drawings illustrate exemplary embodiments and should not be taken as limiting, with the claims defining the scope of the present description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Referring now to FIG. 1, a wound dressing 10 comprises a carrier film 20 and a hydrocolloid layer 20 in contact with one surface of the carrier film 20. In some embodiments, wound dressing 10 may further comprise a release layer 40 in contact with hydrocolloid layer 20

### Hydrocolloid Adhesive Compositions

In one aspect, there is a hydrocolloid adhesive composition. The hydrocolloid adhesive dressing may comprise a base polymer; a hydrocarbon tackifier, an absorbent and a plasticizer. In some examples, the hydrocarbon tackifier may comprise or consist of a cycloaliphatic hydrocarbon modified with aromatic functional groups. In some examples, the hydrocolloid composition may be translucent or transparent. In some examples, the hydrocolloid composition is transparent.

The hydrocolloid adhesive composition may consist of a base polymer; a hydrocarbon tackifier (optionally, comprising or consisting of a cycloaliphatic hydrocarbon modified with aromatic functional group); an absorbent; and a plasticizer.

The hydrocolloid adhesive composition may comprise or consist of about 5 wt. % to about 20 wt. % base polymer; about 25 wt. % to about 40 wt. % hydrocarbon tackifier, wherein the hydrocarbon tackifier comprises a cycloaliphatic hydrocarbon modified with aromatic functional groups; about 30 wt. % to about 50 wt. % absorbent; and up to about 20 wt. % plasticizer. The hydrocolloid adhesive composition may comprise about 10 to about 15 wt. % base polymer; about 29 to about 34 wt. % hydrocarbon tackifier, wherein the hydrocarbon tackifier is a cycloaliphatic hydrocarbon modified with aromatic functional groups; about 41 to about 46 wt. % absorbent; and about 6 to about 17 wt. % plasticizer. In some examples, the hydrocolloid adhesive composition comprises about 10 wt. % to about 15 wt. % poly(styrene-isoprene-styrene); about 29 wt. % to about 34 wt. % hydrocarbon tackifier (which may comprise or be a cycloaliphatic hydrocarbon modified with aromatic functional groups); about 41 wt. % to about 46 wt. % carboxymethylcellulose; about 5 wt. % to about 10 wt. % mineral oil; and about 1 wt. % to about 7 wt. % liquid rubber (e.g., liquid isoprene rubber).

The hydrocolloid adhesive composition may comprise about 10 to about 15 wt. % base polymer; about 25 to about 35 wt. % hydrocarbon tackifier, wherein the hydrocarbon tackifier is a cycloaliphatic hydrocarbon modified with aromatic functional groups; about 41 to about 46 wt. % absorbent; and about 6 to about 17 wt. % plasticizer. The hydrocolloid adhesive composition may comprise about 10 wt. % to about 15 wt. % poly(styrene-isoprene-styrene); about 25 wt. % to about 35 wt. % hydrocarbon tackifier; about 41 wt. % to about 46 wt. % carboxymethylcellulose; about 5 wt. % to about 10 wt. % mineral oil; and about 1 wt. % to about 8 wt. % liquid rubber (e.g., liquid isoprene rubber).

### Tackifier

The hydrocolloid adhesive composition may comprise a hydrocarbon tackifier. The hydrocarbon tackifier may comprise a hydrocarbon modified with aromatic functional groups. In some examples, the hydrocarbon tackifier may consist of a cycloaliphatic hydrocarbon modified with aromatic functional groups.

In some examples, the cycloaliphatic hydrocarbon modified with aromatic functional groups comprises at least about 5% aromatic protons, for example, at least about 5.5%, at least about 6%, at least about 6.5%, at least about 7%, at least about 7.5%, at least about 8%, at least about 8.5%, at least about 9%, at least about 9.5%, at least about 10%, at least about 10.5%, at least about 11%, at least about 11.5%, at least about 12%, at least about 12.5%, at least about 13%, at least about 13.5%, at least about 14%, at least about 14.5%, or at least about 15% aromatic protons. In some examples, the cycloaliphatic hydrocarbon modified with aromatic functional groups comprises up to about 15% aromatic protons, for example, up to about 14.5%, up to about 14%, up to about 13.5%, up to about 13%, up to about 12.5%, up to about 12%, up to about 11.5%, up to about 11%, up to about 10.5%, up to about 10%, up to about 9.5%, up to about 9%, up to about 8.5%, up to about 8%, up to about 7.5%, up to about 7%, up to about 6.5%, up to about 6%, up to about 5.5%, or up to about 5% aromatic protons. In some examples, the cycloaliphatic hydrocarbon modified with aromatic functional groups comprises from about 5 wt. % to about 15 wt. % aromatic protons, for example, about 5.5% to about 14.5%, about 6% to about 14%, about 6.5% to about 13.5%, about 7% to about 13%, about 7.5% to about 12.5%, about 8% to about 12%, about 8.5% to about 11.5%, about 9% to about 11%, about 9.5% to about 10.5%, or about 10% to about 10.5% aromatic protons. In some examples the cycloaliphatic hydrocarbon modified with aromatic functional groups comprises from about 9.5% to about 12% aromatic protons. In some examples, percentage of aromatic protons may be measured by using ASTM D5292-99.

In some examples, the hydrocarbon tackifier (e.g., the cycloaliphatic hydrocarbon modified with aromatic functional groups) has a softening point of at least about 70° C., for example, at least about 75° C., at least about 80° C., at least about 85° C., at least about 90° C., at least about 95° C., at least about 100° C., at least about 105° C., or at least about 110° C. In some examples, the hydrocarbon tackifier (e.g., the cycloaliphatic hydrocarbon modified with aromatic functional groups) has a softening point of up to about 110° C., for example, up to about 105° C., up to about 100° C., up to about 95° C., up to about 90° C., up to about 85° C., up to about 80° C. In some examples, the hydrocarbon tackifier (e.g., the cycloaliphatic hydrocarbon modified with aromatic functional groups) has a softening point of from about 70° C. to about 110° C., for example, from about 75° C. to about 105° C., form about 80° C. to about 100° C., from about 85° C. to about 95° C., from about 87° C. to about 93° C., or from about 85° C. to about 95° C. In some examples, the softening point may be the ring-and-ball softening point. In some examples, the softening point may be measured following ASTM E 28.

In some examples, the tackifier (e.g., the cycloaliphatic hydrocarbon modified with aromatic functional groups) has a number average molecular weight of at least about 300 g/mol, for example, at least about 325 g/mol, at least about 350 g/mol, at least about 375 g/mol, at least about 400 g/mol, at least about 425 g/mol, at least about 450 g/mol, at least about 475 g/mol, at least about 500 g/mol, at least about 525 g/mol, at least about 550 g/mol, at least about 575 g/mol, or at least about 600 g/mol. In some examples, the tackifier (e.g., the cycloaliphatic hydrocarbon modified with aromatic functional groups) has a number average molecular weight of up to about 600 g/mol, for example, up to about 575 g/mol, up to about 550 g/mol, up to about 525 g/mol, up to about 500 g/mol, up to about 475 g/mol, up to about 450 g/mol, up to about 425 g/mol, up to about 400 g/mol, up to about 375 g/mol, up to about 350 g/mol, up to about 325 g/mol, or up to about 300 g/mol. In some examples, the tackifier (e.g., the cycloaliphatic hydrocarbon modified with aromatic functional groups) has a number average molecular weight of from about 300 g/mol to about 600 g/mol, for example, about 325 g/mol to about 575 g/mol, about 350 g/mol to about 550 g/mol, about 375 g/mol to about 525 g/mol, about 400 g/mol to about 500 g/mol, about 425 g/mol to about 475 g/mol, about 450 g/mol to about 500 g/mol. The number average molecular weight may be measured by gel permeation chromatography.

In some examples, the tackifier (e.g., the cycloaliphatic hydrocarbon modified with aromatic functional groups) has a weight average molecular weight of at least about 600 g/mol, for example, at least about 625 g/mol, at least about 650 g/mol, at least about 675 g/mol, at least about 700 g/mol, at least about 725 g/mol, at least about 750 g/mol, at least about 775 g/mol, or at least about 800 g/mol. In some examples, the tackifier (e.g., the cycloaliphatic hydrocarbon modified with aromatic functional groups) has a weight average molecular weight of up to about 800 g/mol, for example, up to about 800 g/mol, up to about 775 g/mol, up to about 750 g/mol, up to about 750 g/mol, up to about 725 g/mol, up to about 700 g/mol, up to about 675 g/mol, up to about 650 g/mol, up to about 625 g/mol, or up to about 600 g/mol. In some examples, the tackifier (e.g., the cycloaliphatic hydrocarbon modified with aromatic functional groups) has a weight average molecular weight of from about 600 g/mol to about 800 g/mol, for example, from about 625 g/mol to about 800 g/mol, about 650 g/mol to about 775 g/mol, about 675 g/mol to about 750 g/mol, about 700 g/mol to about 760 g/mol, or about 700 g/mol to about 725 g/mol. The weight average molecular weight may be measured by static light scattering.

The tackifier may comprise a cycloaliphatic hydrocarbon modified with aromatic functional groups. The cycloaliphatic hydrocarbon modified with aromatic functional groups may be a cycloaliphatic hydrocarbon resin modified with aromatic functional groups. The cycloaliphatic hydrocarbon resin may be a C3 to C10 cycloaliphatic hydrocarbon resin, for example, a C5, C6, C7, C8, C9 or C10 cycloaliphatic hydrocarbon resin. In some examples, the cycloaliphatic hydrocarbon resin may be a C5/C9 cycloaliphatic hydrocarbon resin. The aromatic functional groups may be C5 to C10 aromatic functional groups, for example, C5 to C6 aromatic functional groups.

The hydrocolloid adhesive composition may comprise at least about 25 wt. % hydrocarbon tackifier, for example, at least about 26 wt. %, at least about 27 wt. %, at least about 28 wt. %, at least about 29 wt. %, at least about 30 wt. %, at least about 31 wt. %, at least about 32 wt. %, at least about 33 wt. %, at least about 34 wt. %, at least about 35 wt. %, at least about 36 wt. %, at least about 37 wt. %, at least about 38 wt. %, at least about 39 wt. %, or at least about 40 wt. % hydrocarbon tackifier. In some examples, the hydrocolloid adhesive composition may comprise up to about 40 wt. % hydrocarbon tackifier, for example, up to about 39 wt. %, up to about 38 wt. %, up to about 37 wt. %, up to about 36 wt. %, up to about 35 wt. %, up to about 34 wt. %, up to about 33 wt. %, up to about 32 wt. %, up to about 31 wt. %, up to about 30 wt. %, up to about 29 wt. %, up to about 28 wt. %, up to about 27 wt. %, up to about 26 wt. %, or up to about 25 wt. % hydrocarbon tackifier. In some examples, the hydrocolloid adhesive composition may comprise from about 25 wt. % to about 40 wt. % hydrocarbon tackifier, for example, about 26 wt. % to about 39 wt. %, about 27 wt. % to about 38 wt. %, about 28 wt. % to about 37 wt. %, about 29 wt. % to about 36 wt. %, about 30 wt. % to about 35 wt. %, about 31 wt. % to about 34 wt. %, or about 32 wt. % to about 33 wt. % hydrocarbon tackifier. In some examples, the hydrocarbon tackifier comprises or consists of the cycloaliphatic hydrocarbon modified with aromatic functional groups.

### Base Polymer

The hydrocolloid adhesive composition comprises a base polymer. In some examples, the base polymer comprises a rubber based polymer, a block copolymer (for example, a triblock copolymer) or a combination thereof. In some examples, the base polymer comprises isobutylene, epichlorohydrin rubber, chloroprene rubber, isoprene rubber, bromobutyl rubber, chlorobutyl rubber, poly(styrene-olefin-styrene), poly(ethylene vinyl acetate) or a combination thereof. In some examples, the poly(styrene-olefin-styrene) is selected from poly(styrene-isoprene-styrene), poly(styrene-butadiene-styrene), poly(styrene-ethylene-butylene-styrene), poly(styrene-ethylene-styrene), poly(styrene-propylene-styrene), or combinations thereof. In some examples, the base polymer comprises or consists of poly(styrene-isoprene-styrene). Examples of suitable base polymers include the Kraton™ series of polymers from Shell Chemicals (e.g., Kraton™ D1107 and Kraton™ D1100) and YH-1105 from Sinopec.

The hydrocolloid adhesive composition may comprise at least about 5 wt. % base polymer, for example, at least about 11 wt. %, at least about 12 wt. %, at least about 13 wt. %, at least about 14 wt. %, at least about 15 wt. %, at least about 16 wt. %, at least about 17 wt. %, at least about 18 wt. %, at least about 19 wt. %, or at least about 20 wt. % base polymer. In some examples, the hydrocolloid adhesive composition may comprise up to about 20 wt. % base polymer, for example, up to about 19 wt. %, up to about 18 wt. %, up to about 17 wt. %, up to about 16 wt. %, up to about 15 wt. %, up to about 14 wt. %, up to about 13 wt. %, up to about 12 wt. %, up to about 11 wt. %, up to about 10 wt. %, up to about 9 wt. %, up to about 8 wt. %, up to about 7 wt. %, up to about 6 wt. %, or up to about 5 wt. % base polymer. In some examples, the hydrocolloid adhesive composition comprises from about 5 wt. % to about 20 wt. % base polymer, for example, about 6 wt. % to about 19 wt. %, about 7 wt. % to about 18 wt. %, about 8 wt. % to about 17 wt. %, about 9 wt. % to about 16 wt. %, about 10 wt. % to about 15 wt. %, about 11 wt. % to about 14 wt. %, or about 12 wt. % to about 13 wt. % base polymer.

### Absorbent

The hydrocolloid adhesive composition may comprise an absorbent. In some examples, the absorbent is a hydrophilic polymer. In some examples, the absorbent may comprise pectin, alginates, natural plant gums such as karaya gum, guar cum, gum arabic, cellulose derivatives, sodium starch clycolate, polyvinyl alcohol, polyethylene glycol, gelatin, carboxypolymethylene, carboxymethyl starches, carrageenan, or combinations thereof. In some examples, the absorbent may comprise or consist of cellulose derivatives such as carboxymethylcellulose, hydroxyethylcellulose, hydroxypropylcellulse, methylcellulose, hydroxypropylmethylcellulose and their alkali metal salts (e.g., sodium or calcium). In some examples, the absorbent may comprise or consist of a cellulose derivative or a mixture of cellulose derivatives. In some examples, the absorbent may comprise or consist of carboxymethylcellulose or an alkali metal salt thereof. In some examples, the absorbent may comprise or consist of an alkali metal salt of carboxymethylcellulose, for example, sodium carboxymethylcellulose.

The hydrocolloid adhesive composition may comprise at least about 30 wt. % absorbent, for example, at least about 31 wt. %, at least about 32 wt. %, at least about 33 wt. %, at least about 34 wt. %, at least about 35 wt. %, at least about 36 wt. %, at least about 37 wt. %, at least about 38 wt. %, at least about 39 wt. %, at least about 40 wt. %, at least about 41 wt. %, at least about 42 wt. %, at least about 43 wt. %, at least about 44 wt. %, at least about 45 wt. %, at least about 46 wt. %, at least about 47 wt. %, at least about 48 wt. %, at least about 49 wt. %, or at least about 50 wt. % absorbent. In some examples, the hydrocolloid adhesive composition may comprise up to about 50 wt. % absorbent, for example, up to about 49 wt. %, up to about 48 wt. %, up to about 47 wt. %, up to about 46 wt. %, up to about 45 wt. %, up to about 44 wt. %, up to about 43 wt. %, up to about 42 wt. %, up to about 41 wt. %, up to about 40 wt. %, up to about 39 wt. %, up to about 38 wt. %, up to about 37 wt. %, up to about 36 wt. %, up to about 35 wt. %, up to about 34 wt. %, up to about 33 wt. %, up to about 32 wt. %, up to about 31 wt. %, or up to about 30 wt. %. In some examples, the hydrocolloid adhesive composition may comprise from about 30 wt. % to about 50 wt. % absorbent, for example, about 31 wt. % to about 50 wt. %, about 32 wt. % to about 49 wt. %, about 33 wt. % to about 48 wt. %, about 34 wt. % to about 47 wt. %, about 35 wt. % to about 46 wt. %, about 36 wt. % to about 45 wt. %, about 37 wt. % to about 44 wt. %, about 38 wt. % to about 43 wt. %, about 39 wt. % to about 42 wt. %, or about 40 wt. % to about 41 wt. % absorbent.

### Plasticizer

The hydrocolloid adhesive composition may comprise a plasticizer. In some examples, the hydrocolloid adhesive composition may comprise at least two plasticizers, for example, two plasticizers.

In some examples, the plasticizer may comprise a plasticizing oil, for example, mineral oil or vegetable oil. In some examples, the plasticizer may comprise mineral oil, citrate oil, coconut oil, paraffin oil, phthalic acid esters, adipic acid esters, liquid or solid resins, liquid rubber (for example, liquid isoprene rubber and/or liquid butadiene rubber) or combinations thereof. In some examples, the plasticizer may comprise or consist of mineral oil, liquid isoprene rubber or combinations thereof. In some examples, the plasticizer may comprise or consist of mineral oil and liquid rubber (e.g., liquid isoprene rubber).

The hydrocolloid adhesive composition may comprise up to about 20 wt. % plasticizer, for example, up to about 19 wt. %, up to about 18 wt. %, up to about 17 wt. %, up to about 16 wt. %, up to about 15 wt. %, up to about 14 wt. %, up to about 13 wt. %, up to about 12 wt. %, up to about 11 wt. %, up to about 10 wt. %, up to about 9 wt. %, up to about 8 wt. %, up to about 7 wt. %, up to about 6 wt. %, up to about 5 wt. %, up to about 4 wt. %, up to about 3 wt. %, up to about 2 wt. %, or up to about 1 wt. %. In some examples, the hydrocolloid adhesive composition may comprise at least about 1 wt. % plasticizer, for example, at least about 2 wt. %, at least about 3 wt. %, at least about 4 wt. %, at least about 5 wt. %, at least about 6 wt. %, at least about 7 wt. %, at least about 8 wt. %, at least about 9 wt. %, at least about 10 wt. %, at least about 11 wt. %, at least about 12 wt. %, at least about 13 wt. %, at least about 14 wt. %, at least about 15 wt. %, at least about 16 wt. %, at least about 17 wt. %, at least about 18 wt. %, at least about 19 wt. %, or at least about 20 wt. % plasticizer. In some examples, the hydrocolloid adhesive composition comprises from about 1 wt. % to about 20 wt. % plasticizer, for example, about 2 wt. % to about 19 wt. %, about 3 wt. % to about 18 wt. %, about 4 wt. % to about 17 wt. %, about 5 wt. % to about 16 wt. %, about 6 wt. % to about 15 wt. %, about 7 wt. % to about 14 wt. %, about 8 wt. % to about 13 wt. %, about 9 wt. % to about 12 wt. %, or about 10 wt. % to about 11 wt. % plasticizer.

In some examples, the hydrocolloid adhesive composition comprises mineral oil and liquid rubber (e.g., liquid isoprene rubber). In some examples, the hydrocolloid adhesive composition comprises from about 5 wt. % to about 10 wt. % mineral oil and from about 0 wt. % to about 8 wt. % liquid rubber (e.g., liquid isoprene rubber). In some examples, the hydrocolloid adhesive composition comprises from about 5 wt. % to about 10 wt. % mineral oil; and from about 1 wt. % to about 8 wt. %, for example, about 1 wt. % to about 7 wt. % liquid rubber (e.g., liquid isoprene rubber).

### Wound Dressing

In another aspect, there is provided a wound dressing. The wound dressing may comprise a carrier film and a hydrocolloid layer disposed on the carrier film. The hydrocolloid layer may comprise or consist of any hydrocolloid adhesive composition described herein. In some examples, the hydrocolloid layer may consist of any hydrocolloid adhesive composition described herein.

The hydrocolloid layer may be a continuous or discontinuous layer of hydrocolloid adhesive composition. In some examples, the wound dressing comprises a continuous hydrocolloid layer disposed on a carrier film. In some examples, the wound dressing comprises a discontinuous hydrocolloid layer disposed on a carrier film.

The adhesive dressing may comprise a discontinuous hydrocolloid layer, wherein the hydrocolloid adhesive composition form any shape on the carrier film. In some examples, the adhesive dressing may comprise a discontinuous hydrocolloid layer, wherein the discontinuous hydrocolloid layer may comprise a plurality of lines of hydrocolloid, a plurality of dots of hydrocolloid, or a combination thereof. In some examples, the plurality of lines of hydrocolloid may be undulating lines of hydrocolloid, straight lines of hydrocolloid, parallel lines of hydrocolloid, parallel straight lines of hydrocolloid, parallel undulating lines of hydrocolloid or a combination thereof.

In some examples, the wound dressing may comprise a discontinuous hydrocolloid adhesive composition disposed on a carrier film, wherein the hydrocolloid adhesive composition forms undulating lines, for example, parallel undulating lines of hydrocolloid adhesive composition on the carrier film. The undulating lines of hydrocolloid may be sinusoidal. The undulating lines of hydrocolloid may have an amplitude and a frequency. In some examples, the wound dressing may comprise a plurality of undulating lines of hydrocolloid disposed on the carrier film, a plurality of dots disposed on the carrier film, a plurality of parallel lines of hydrocolloid disposed on the hydrocolloid, a plurality of undulating parallel lines of hydrocolloid disposed on the carrier film, or a combination thereof.

In some examples, the wound dressing may comprise first portion comprising a discontinuous hydrocolloid layer and a second portion comprising a continuous hydrocolloid layer. In some examples, the second portion may surround the first portion. Thus, the wound dressing may comprise a border of hydrocolloid adhesive composition surrounding a central area in which the hydrocolloid adhesive composition is disposed in a discontinuous pattern.

In some examples, the wound dressing may comprise any suitable carrier film. In The carrier film may be a polymeric film. The carrier film may form the external surface of the wound dressing, that is, the non-wound contacting surface of the wound dressing. In some examples, the carrier film comprises or consists of a polyurethane film, an ethylene vinyl acetate film, a polyethylene film, an ethylene propylene elastomer film, a styrene-ethylene-butadiene-styrene film or a combination thereof. In some examples, the carrier film comprises or consists of a polyurethane film, an ethylene vinyl acetate film or a combination thereof. In some examples, the carrier film comprises or consists of a polyurethane film. In some examples, the carrier film comprises or consists of an ethylene vinyl acetate film.

In some examples, the wound dressing is translucent or transparent. In some examples, the wound dressing is at least partially transparent, enabling observation of a wound through the wound dressing. In some examples, the carrier film is translucent or transparent. In some examples, the hydrocolloid layer is translucent or transparent. In some examples, the carrier film and the hydrocolloid layer are both translucent and transparent. “Transparent” as used herein means that the material transmits incident light with negligible scattering and little absorption, enabling a wound to be seen through the transparent wound dressing (or the transparent part of the wound dressing) under typical, unaided viewing conditions. This may, for example, be judged by whether words of size 12 text in Times New Roman font printed on white paper can be read through the transparent wound dressing or transparent part of a wound dressing.

This may, for example, be judged by refractive index. For example, the transparent wound dressing may have a refractive index ranging from about 1 to about 2. For example, the refractive index may be equal to or less than about 1.9 or equal to or less than 1.8 or equal to or less than 1.7. the refractive index may be measured using a refractometer.

Alternatively or additionally, this may be judged by percentage light transmission, for example, using a transparency meter. A translucent or transparent wound dressing (or part of a wound dressing) may have a total transmittance of at least about 50%, for example, at least about 55%, at least about 60%, at least about 65%, at least about 70%, at least about 75%, at least about 80%, at least about 85%, at least about 90%.

In some examples, the wound dressing may comprise an additional adhesive disposed between the carrier film and the hydrocolloid layer. In other examples, the carrier film may be in contact with the hydrocolloid layer.

In some examples, the additional adhesive is translucent or transparent. In some examples, the wound dressing comprising the additional adhesive is translucent or transparent. In some examples, the additional adhesive is an acrylic adhesive.

The wound dressing may additionally comprise a release liner. The release liner is removed from the wound dressing before application to a wound. The release liner may contact the hydrocolloid layer. The release liner is positioned on the outermost wound-facing side of the wound dressing. The release liner facilitates ease of handling and packaging and may, for example, assist in maintaining the wound dressing in a sterile condition. The release liner may, for example, cover the entire surface of the wound dressing.

The release liner may, for example, comprise a silicone surface. In some examples, the release liner may be a paper substrate coated with a release composition. In some examples, the release liner comprises a silicon coated paper or plastic release liner.

In some examples, the hydrocolloid layer has a thickness of up to about 5 mm, for example, up to about 4 mm, up to about 3 mm, up to about 2 mm, up to about 1.5 mm, up to about 1 mm, up to about 0.9 mm, up to about 0.8 mm, up to about 0.7 mm, up to about 0.6 mm, up to about 0.5 mm, up to about 0.4 mm, up to about 0.3 mm, up to about 0.2 mm. In some examples, the hydrocolloid layer has a thickness of from about 0.3 mm to about 5 mm, for example, about 0.6 mm to about 2.5 mm, about 0.7 mm to about 2 mm, about 0.8 mm, to about 1.5 mm, or about 0.9 mm to about 1 mm.

In some examples, the coat weight of the hydrocolloid adhesive composition may be from about 300 g/m² to about 5600 g/m², for example, from about 300 g/m² to about 5000 g/m², from about 400 g/m² to about 4000 g/m², from about 500 g/m² to about 3000 g/m², from about 600 g/m² to about 2000 g/m², from about 700 g/m² to about 1000 g/m².

In some examples, the carrier film may have a moisture vapor transmission rate (MVTR) of at least about 100 g/m³/24 hours, optionally, at least about 200 g/m²/24 hours, at least about 500 g/m²/24 hours, or at least about 600 g/m²/24 hours. The carrier film may have an MVTR of from about 100 g/m²/24 hours to about 3000 g/m²/24 hours, or from about 1000 g/m²/24 hours to about 3000 g/m²/24 hours. The MVTR may be measured in accordance with BS EN 13726-2:2002.

In some examples, the carrier film may have a thickness ranging from about 10 μm to about 100 μm, for example, about 20 μm to about 90 μm, about 10 μm to about 50 μm.

### Method of Producing a Wound Dressing

In a further aspect, there is provided a method of producing a wound dressing. The method of producing a wound dressing may comprise depositing a hydrocolloid adhesive composition onto a carrier film to form a hydrocolloid layer. The hydrocolloid adhesive composition may be any hydrocolloid adhesive composition described herein. The method of producing a wound dressing may produce any wound dressing described herein.

In some examples, the method of producing a wound dressing may comprise depositing an additional adhesive on a carrier film and then depositing a hydrocolloid adhesive composition on the additional adhesive disposed on the carrier film.

In some examples, the method of producing a wound dressing may comprise depositing a discontinuous hydrocolloid layer onto a carrier film. In other examples, the method of producing a wound dressing may comprise depositing a continuous hydrocolloid layer onto a carrier film. In some examples, the method of producing a wound dressing may comprise depositing a continuous hydrocolloid layer onto a carrier film and removing a portion of the hydrocolloid layer to form a discontinuous hydrocolloid layer.

In some examples, the method of producing a wound dressing comprises laminating the carrier film and the hydrocolloid adhesive composition. In some example, the carrier film is laminated to the hydrocolloid adhesive composition at a temperature of at least about 50° C., at least about 60° C., at least about 70° C., at least about 75° C., at least about 80° C. In some examples, the carrier film is laminated to the hydrocolloid adhesive composition at a temperature of up to about 110° C., for example, up to about 105° C., up to about 100° C., up to about 95° C., up to about 90° C., up to about 85° C., up to about 80° C. in some examples, the carrier film is laminated to the hydrocolloid adhesive composition at a temperature of about 50° C. to about 110° C., for example, about 55° C. to about 105° C., about 60° C. to about 100° C., about 65° C. to about 105° C., about 70° C. to about 100° C., about 75° C. to about 95° C., about 80° C. to about 90° C., about 80° C. to about 85° C. In some examples, the carrier film is laminated to the hydrocolloid adhesive composition at a pressure of about 5 KPSI to about 15 KPSI, for example, about 6 KPSI to about 14 KPSI, about 7 KPSI to about 13 KPSI, about 8 KPSI to about 12 KPSI, about 9 KPSI to about 11 KPSI, about 10 KPSO to about 11 KPSI.

In some examples, the method of producing a wound dressing comprises depositing a hydrocolloid adhesive composition onto a carrier film to form a hydrocolloid layer and contacting a release liner with the hydrocolloid layer. In some examples, the method of producing a wound dressing comprises depositing an additional adhesive onto a carrier film and then depositing a hydrocolloid adhesive composition onto the additional adhesive to form a hydrocolloid layer. In some examples, a release liner is then contacted with the hydrocolloid layer.

In some examples, the method of producing a wound dressing may comprise forming a hydrocolloid adhesive composition and depositing the hydrocolloid adhesive composition on a carrier film to form a hydrocolloid layer.

In some examples, a hydrocolloid adhesive composition may be formed by combining a base polymer, a hydrocarbon tackifier (e.g., a hydrocarbon tackifier comprising a cycloaliphatic hydrocarbon modified with aromatic functional groups), an absorbent and a plasticizer. In some examples, the components of the hydrocolloid adhesive composition may be combined in any order.

In some examples, the hydrocolloid adhesive composition may be formed by combining a base polymer (e.g., styrene-olefin-styrene) with a plasticizer (e.g., mineral oil, liquid isoprene rubber, or a combination thereof) to form a first composition; adding the hydrocarbon tackifier to form a second composition; and then adding the absorbent (e.g., carboxymethylcellulose) to form the hydrocolloid adhesive composition. In some examples, the hydrocolloid adhesive composition may be formed by combining a base polymer with a plasticizer at a first temperature to form a first composition; adding the hydrocarbon tackifier to form a second composition; changing the temperature (e.g., reducing the temperature to, for example, 104° C.); and adding the absorbent. In some examples, after addition of the absorbent, the composition is cooled (actively or passively), for example, to room temperature.

In some examples, the first temperature may be at least about 110° C., for example, at least about 115° C., at least about 120° C., at least about 125° C., at least about 130° C., at least about 135° C., at least about 138° C. In some examples, the first temperature may be up to about 175° C., for example, up to about 170° C., up to about 165° C., up to about 160° C., up to about 155° C., up to about 150° C., up to about 145° C., up to about 140° C. In some examples, the first temperature may be from about 110° C. to about 175° C., for example, about 115° C. to about 170° C., about 120° C. to about 165° C., about 125° C. to about 160° C., about 130° C. to about 155° C., about 135° C. to about 150° C., about 137° C. to about 145° C. In some examples, the second temperature may be at least about 75° C., for example, at least about 80° C., at least about 85° C., at least about 90° C., at least about 95° C., at least about 100° C. In some examples, the second temperature may be up to about 125° C., for example, up to about 120° C., up to about 115° C., up to about 110° C., or up to about 105° C. In some examples, the second temperature may be about 75° C. to about 125° C., for example, about 85° C. to about 120° C., about 95° C. to about 110° C., or about 100° C. to about 105° C.

In some examples, the first composition may be mixed for up to 1 hour before addition of the hydrocarbon tackifier, for example, from about 30 minutes to 1 hour, about 40 minutes to about 55 minutes, or about 45 minutes to about 50 minutes. In some examples, the second composition is mixed for up to 1 hour, for example, 30 minutes to 1 hour, about 35 minutes to 50 minutes, about 40 minutes to 45 minutes before addition of the absorbent. In some examples, after the addition of the absorbent, the composition may be mixed for up to 1 hour to form the hydrocolloid adhesive composition, for example, from 30 minutes to 1 hour, 35 minutes to 55 minutes, or 40 minutes to 45 minutes. In some examples, after addition of the absorbent, the hydrocolloid adhesive composition may be cooled, for example, to room temperature. In some examples, the cooling may be active or passive cooling.

In some examples, the hydrocolloid adhesive composition comprises multiple plasticizers. In some examples, the plasticizers are added to the composition together. In other examples, each plasticizer is added separately.

### EXAMPLES

The following illustrates examples of the methods and other aspects described herein. Thus, these examples should not be considered as limitations of the present description but are merely in place to teach how to make examples of the present description.

### Materials

Materials used in the hydrocolloid adhesive compositions are shown in the following Table 1:

TABLE 1

| | | Reference | Reference | | | | |
|---|---|---|---|---|---|---|---|
| | Example | Example | Example | Example | Example | Example | Example |
| | 1 | 2 | 3 | 4 | 5 | 6 | 7 |
| MATERIALS | wt. % | wt. % | wt. % | wt. % | wt. % | wt. % | wt. % |
| SIS elastomer | 14 | 14 | 14 | 14 | 14 | 14 | 13 |
| Mineral oil | 7 | 7 | 7 | 7 | 7 | 7 | 7 |
| LIR | | | | 3 | 3 | 7 | 5 |
| Escorez 5690 | 34 | | | 32 | 32 | 27 | 32 |
| EastoTac H100W | | 34 | | | | | |
| Wingtack Plus | | | 34 | | | | |
| CMC | 45 | 45 | 45 | 44 | 44 | 45 | 43 |
| Total | 100 | 100 | 100 | 100 | 100 | 100 | 100 |

Thermoplastic Elastomer (base polymer): Synopec SIS YH-1106 (styrene-isoprene-styrene block copolymer) with a melt index in the range of 8-14 g/10 min available from BEC Materials, China.

Mineral Oil: Citation 350 with a kinematic viscosity in the range of 63-71 cSt manufactured by Avatar Corporation, USA

LIR: Liquid isoprene rubber with viscosity range 28K cps-55K cps were used in the formulations. Trade names: LIR-30 obtained from Kuraray, USA and Isolene 40S from H.B. Fuller, USA. LIR-30 was used in Examples 4 and 7 and Isolene 40S was used Examples 5 and 6.

Tackifier: Different hydrocarbon resin based tackifiers were used in the adhesive formulations. Escorez 5690 is an aromatic modified, cycloaliphatic hydrocarbon resin available from ExxonMobil, USA. Typical properties of Escorez 5690 are: softening point: 90.5° C., melt viscosity (at 160° C.): 350 mPa s, a number average molecular weight: 450 g/mol, a weight average molecular weight: 760 g/mol, glass transition temperature: 44° C. and aromaticity: 10.1% (% of aromatic protons). Eastotac H100W is a hydrogenated hydrocarbon resin available from Eastman, USA. Typical properties of Eastotac H100W are (unless mentioned otherwise, measured at 23° C. and 50% humidity and by the relevant ASTM): softening point: 100° C. (ASTM E 28); density: 1.04 g/m; Brookfield viscosity (190° C.): 200 cP; glass transition temperature (differential scanning calorimetry): 47° C.; number average molecular weight: 500; weight average molecular weight: 930. Wingtac Plus is an aromatically modified C-5 hydrocarbon resin available from Cay Valley, USA. Typical properties of Wingtac Plus are: softening point: 96° C.; specific gravity (25° C.): 0.95; glass transition temperature: 53° C. (midpoint) and 47° C. (onset); number average molecular weight: 1000; weight average molecular weight: 1600.

Absorbent: Sodium carboxymethyl cellulose was used as the absorbent. Specifically, Gelycel F1-6000 (available from AMTEX, USA) and Aqualon (available from Ashland, USA) were used in Example 4 and Example 5 respectively, while Finsin H5 (available from SynoCMC, China) was used for all other examples.

### Method of Producing the Wound Dressing

### Mixing Procedure

400 g of the hydrocolloid adhesive composition was combined in a laboratory Z-blade mixer (ID #ELABE53), heating at 280° F. (138° C.) and using a mixing speed of 60 rpm. All ingredients of the hydrocolloid adhesive composition were weighed using a laboratory balance as per the formulation requirements. The following sequence was followed in making all adhesive compositions (Example 1-7) as shown in the table.

The thermoplastic elastomer (also referred to herein as the base polymer), mineral oil (a plasticizer), and, if present, liquid isoprene rubber (a plasticizer) were added to the mixer first and mixed for about 50 min to make a homogeneous blend. Then, the hydrocarbon tackifier was added to the mixer and mixed for about 45 min. Next, the temperature of the mixer was changed to 220° F. (104° C.), maintaining the Z-blade rotor speed the same. The CMC was then added to the mixer and mixed about 45 min. After the mixing process the adhesive was collected and cooled for further processing for making test samples.

### Lamination/Pressing

The lamination was carried out on laboratory carver press. Stainless steel strips were used to make the laminates with the desired adhesive thickness (0.7-1.0 mm), making sure that the paper liner (release liner) and polyurethane carrier film extend over the stainless-steel strips on both sides between the press platens. The press platen temperature was set at 80° C. and a pressure of 10 KPSI with a dwell time of 10 s. Laminates from each hydrocolloid adhesive composition were made by pressing about 20 g of the hydrocolloid adhesive composition between a silicone-coated paper release liner (obtained from Rayven/R32-12; 170 micron polycoated-paper) and a polyurethane film (carrier film) with a paper carrier (obtained from Windsor/R21-18; 30 micron polyurethane film on a 170 mm paper carrier).

### Testing

Test samples were prepared by die-cutting specimens from the pressed laminate after removing the paper carrier from the polyurethane carrier film. The construction of the laminates during testing is shown in FIG. 1. The adhesive performance was evaluated as per ASTM methods or standard Euromed laboratory methods.

### Absorption Test

An absorption test was conducted to measure the fluid handing capability of the hydrocolloid adhesive compositions over a set interval of time. To perform this test first saline water is prepared (0.9 wt. % ACS grade sodium chloride, NaCl) using a mixture of 34.2 g of NaCl per 1 US gallon of distilled water. A circular disc with a diameter of 1 inch of the test article was die cut from the laminate. Place a PET tape with adhesive on the PU carrier film of the test article and remove the release liner. Weigh the test article (W1) and then submerged in the saline water in a well (˜60 ml) at room temperature (22° C.). The saline water should be used in a sufficient so that the test article is completely immersed, and not floating. After 24 hours of submersion, the specimens were taken out of the saline water with silicone tipped forceps (gently tapped a few times to remove excess fluid from the sample) and weighed (W2). The water absorption was calculated based on the following equation and reported as a percentage.

$$Absorption [ \% ] = ( W 2 - W 1 ) / W 1 \times 100$$

in which:
W1=Weight of test article before submersion in saline water
W2=Weight of test article after 24 hrs of submersion in saline water

### Liner Release Test

The liner release test measures the release force between the release liner and the hydrocolloid adhesive composition. The test specimen is cut to a length of 50 mm (+/−1.5 mm) and width of 55 mm (+/−1.5 mm). The liner release test is performed at a 180° angle on a tensile tester using a Mark 10 digital force gauge.

The test article is fixed to the tester by clamping the release liner to the top steel clamp (movable) and the hydrocolloid adhesive and carrier film side of the test article to the bottom steel clamp (fixed). The peeling test is performed at an upper clamp speed of 300 mm/min and the force gauge measures in Newton. The specimen must be held perpendicular to the plate clamps and not at an angle during the testing. The average liner release force (N) is calculated after removing the first and last 5% of the results.

### Peel Adhesion Test

The peel adhesion test measures the force required to remove the pressure sensitive adhesive/the hydrocolloid adhesive composition from 304 stainless steel test panel at a 90° angle and a standard test speed of 5.0 mm/s in accordance of ASTM A666. The test article is cut to form a 25 mm (+/−1.5 mm) wide and 100 mm (+/−1.5 mm) length shape. The stainless-steel test panels are 5 mm (+/−1.5 mm) by 150 mm (1.5 mm) and are cleaned with alcohol after each use and allowed to dry for 5 minutes.

The test article is removed from its liner and placed onto the stainless steel test panel, keeping 20-30 mm of release liner attached at one end of the test article. The test article is then attached on the test panel by rolling a roller (2.64 kg (+/−0.1 kg)), once forward and once backward in each lengthwise direction. The test article is then affixed to the tensile tester and the sample is peeled. The speed of puling is 300 mm/min and data recorded at approximately 3.3 Hz. The average of peel force is calculated after removing the first and last 5% of the results.

### Probe Tack Test

The tack test method is used to measure the tack characteristics of adhesive materials. The tack is a measure of force required to separate the adhesive from a surface shortly after they have been brought into contact. The test is carried out on a circular test article (1 inch diameter) using a calibrated PT-1000 probe tack tester. The test area of the adhesive should be smooth and free of indentations. The test probe (5 mm diameter) and the annular ring (applies a force of 9.79+/−0.1 KPa) are cleaned with a lint free wipe containing alcohol and allowed to dry for 10 minutes.

The test article is used after removing the release liner and placing the adhesive side of the test article on the annular ring in the Test Platform. After test initiation, the test platform moves downward and maintains probe contact with the sample for a specified time (1 sec). The test platform then moves upward at a speed of 24 in/min during which time the peak force is measured, and recorded as the probe tack force in grams.

### Modulus Test

The modulus test is performed to determine how flexible and comfortable the product will be for a patient. The test is carried out on a 25 mm (+/−11.5 mm) width by 50 mm (+/−1.5 mm) length specimen using a Mark 10 tensile tester fitted with a digital force gauge. The test clamps are set to 25 mm apart and the test specimen is fixed by tape to the upper and bottom clamps of the tensile tester. The modulus testing is conducted at a clamp speed of 300 mm/min. The Modulus is the force at 100% elongation of the specimen and reported in Newton. The results are shown below in Table 2.

TABLE 2

| | | Example | Reference | Reference | Example | Example | Example | Example |
|---|---|---|---|---|---|---|---|---|
| PROPERTIES | | 1 | Example 2 | Example 3 | 4 | 5 | 6 | 7 |
| Absorption, | % | 566 | 871 | 853 | 656 | 618 | 316 | 546 |
| wt. % | | | | | | | | |
| Release from | N | 0.49 | 0.71 | 1.19 | 0.4 | 0.72 | 0.72 | 1.15 |
| Liner | | | | | | | | |
| Peel Adhesion | N | 7.5 | 8.1 | 9.7 | 6.3 | 6.7 | 4.29 | 5.6 |
| Probe Tack | g | 494 | 307 | 349 | 449 | 372 | 324 | 361 |
| Modulus | N | 8.6 | 14.9 | 11.6 | 4.4 | 7.4 | 5.0 | 6.0 |

As can be seen from these results, wound dressings in which the hydrocolloid adhesive composition contains EastoTac H100W or Wingtack Plus have a modulus that is too high for use in wound dressings. Moreover, wound dressings of Examples 1 and 4 to 7 have greater transparency than other hydrocolloid wound dressings. Indeed, wound dressings containing colophony generally are generally yellow in color. In contrast, hydrocolloids of Examples 1 and 4 to 7 are clear and colorless, allowing a wound to be viewed through the hydrocolloid adhesive layer. When combined with a clear carrier film, such as a polyurethane or ethylene vinyl acetate carrier film, a wound can be seen through the wound dressing.

Additionally, hydrocolloid adhesive compositions of Examples 1 and 4 to 7 have low reactivity. As a result, these hydrocolloid adhesive compositions can be combined with carrier films such as ethylene vinyl acetate carrier films that react with some previous hydrocolloid adhesive compositions, reducing unwanted shrinkage of carrier films and wound dressings.

Wound dressings according to Examples 1 and 4 to 7 have sufficient adhesion to skin without adhering so strongly that their removal causes significant pain to the patient. These hydrocolloid adhesive compositions also have lower modulus characteristics that allow good conformability of the dressing to the skin and good flexibility that provides comfort to the user.

### Patterned Adhesive Layers

Although wound dressings containing hydrocolloid adhesive layers described herein are initially transparent, when they are used on highly exuding wounds, the transparency of the hydrocolloid adhesive layer can decrease over time due to absorption of colored exudate. Wound dressings in which a discontinuous hydrocolloid adhesive layer is used are transparent for significantly longer, without significantly reducing the absorption capacity of the wound dressing. Moreover, such wound dressings are also more flexible and comfortable for the patient.

In particular, it has been found that wound dressings in which an undulating pattern of hydrocolloid adhesive (e.g., parallel undulating lines of hydrocolloid adhesive) provides a more flexible and comfortable dressing that shows improved transparency even after high absorption of exudate than other patterns of hydrocolloid adhesive composition. Furthermore, this undulating pattern of hydrocolloid adhesive composition may also promote wound healing.

It is noted that, as used in this specification and the appended claims, the singular forms “a,” “an,” and “the” include plural referents unless the context clearly dictates otherwise.

As used herein, “co-polymer” refers to a polymer that is polymerized from at least two monomers.

A certain monomer may be described herein as constituting a certain weight percentage of a polymer. This indicates that the repeating units formed from the said monomer in the polymer constitute said weight percentage of the polymer.

If a standard test is mentioned herein, unless otherwise stated, the version of the test to be referred to is the most recent at the time of filing this patent application.

As used herein, the term “about” is used to provide flexibility to a numerical range endpoint by providing that a given value may be a little above or a little below the endpoint to allow for variation in test methods or apparatus. The degree of flexibility of this term can be dictated by the particular variable and would be within the knowledge of those skilled in the art to determine based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not just the numerical values explicitly recited as the limits of the range, but also to include all the individual numerical values or sub-ranges encompassed within that range as if each numerical value and sub-range is explicitly recited. As an illustration, a numerical range of “about 1 wt. % to about 5 wt. %” should be interpreted to include not just the explicitly recited values of about 1 wt. % to about 5 wt. %, but also to include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting a single numerical value. Furthermore, such an interpretation should apply regardless of the breadth of the range or the characteristics being described.

As used herein, unless otherwise stated, wt. % values are to be taken as referring to a weight-for-weight (w/w) percentage of total composition.

Unless otherwise stated, any feature described herein can be combined with any aspect or any other feature described herein.

While the invention has been described in detail herein in accordance with certain preferred embodiments thereof, many modifications and changes therein may be effected by those skilled in the art. Accordingly, the foregoing description should not be construed to be limited thereby but should be construed to include such aforementioned obvious variations and be limited only by the spirit and scope of the following claims.

For example, in a first aspect, a first embodiment is a hydrocolloid adhesive composition comprising a base polymer, a hydrocarbon tackifier, an absorbent and a plasticizer.

A second embodiment is the first embodiment, wherein the base polymer comprises about 5% to about 20% by weight of the composition.

A third embodiment is any combination of the first two embodiments, wherein the hydrocarbon tackifier comprise about 25% to about 40% by weight of the composition.

A 4^{th} embodiment is any combination of the first 3 embodiments, wherein the absorbent comprises about 30% to about 50% by weight of the composition.

A 5^{th} embodiment is any combination of the first 4 embodiments, wherein the plasticizer comprises up to about 20% by weight of the composition.

A 6^{th} embodiment is any combination of the first 5 embodiments, wherein the hydrocarbon tackifier is a cycloaliphatic hydrocarbon modified with aromatic functional groups.

A 7^{th} embodiment is any combination of the first 6 embodiments, wherein the cycloaliphatic hydrocarbon modified with aromatic functional groups comprises about 5 wt. % to about 15 wt. % aromatic protons.

An 8^{th} embodiment is any combination of the first 7 embodiments, wherein the cycloaliphatic hydrocarbon modified with aromatic functional groups has a softening point of from about 80° C. to about 100° C.

A 9^{th} embodiment is any combination of the first 8 embodiments, wherein the base polymer comprises a poly(styrene-olefin-styrene) polymer or a poly(styrene-isoprene-styrene) polymer.

A 10^{th} embodiment is any combination of the first 9 embodiments, wherein the absorbent is a hydrophilic polymer.

An 11^{th} embodiment is any combination of the first 10 embodiments, wherein the hydrophilic polymer is a cellulose.

A 12^{th} embodiment is any combination of the first 11 embodiments, wherein the plasticizer is selected from mineral oil, liquid isoprene rubber and combinations thereof.

A 13^{th} embodiment is any combination of the first 12 embodiments, further comprising: about 10 to about 15 wt. % base polymer, about 25 to about 35 wt. % hydrocarbon tackifier, wherein the hydrocarbon tackifier is a cycloaliphatic hydrocarbon modified with aromatic functional groups; about 41 to about 46 wt. % absorbent; and about 6 to about 17 wt. % plasticizer.

A 14^{th} embodiment is any combination of the first 13 embodiments, further comprising: about 10 wt. % to about 15 wt. % poly(styrene-isoprene-styrene); about 25 wt. % to about 35 wt. % hydrocarbon tackifier; about 41 wt. % to about 46 wt. % carboxymethylcellulose; about 5 wt. % to about 10 wt. % mineral oil and about 1 wt. % to about 8 wt. % liquid isoprene rubber.

A 15^{th} embodiment is any combination of the first 14 embodiments, wherein the composition has a refractive index ranging from about 1 to about 2.

In another aspect, a first embodiment is a wound dressing comprising a carrier film, and a hydrocolloid layer disposed on the carrier film. The hydrocolloid layer comprises a base polymer, a hydrocarbon tackifier, an absorbent and a plasticizer.

A second embodiment is the first embodiment, wherein the carrier film is selected from the group consisting of a polyurethane film, an ethylene vinyl acetate film, or a combination thereof.

A 3^{rd} embodiment is any combination of the first 2 embodiments, wherein the hydrocolloid layer is discontinuous.

A 4^{th} embodiment is any combination of the first 3 embodiments, wherein the hydrocolloid layer comprises a plurality of undulating lines of the hydrocolloid adhesive composition disposed on the carrier film.

A 5^{th} embodiment is any combination of the first 4 embodiments, wherein the hydrocolloid layer comprises a plurality of dots of the hydrocolloid adhesive composition disposed on the carrier film.

A 6^{th} embodiment is any combination of the first 5 embodiments, wherein the hydrocolloid layer comprises a plurality of parallel lines of the hydrocolloid adhesive composition disposed on the carrier film.

A 7^{th} embodiment is any combination of the first 6 embodiments, wherein the hydrocolloid layer comprises a plurality of undulating parallel lines of the hydrocolloid adhesive composition disposed on the carrier film.

An 8^{th} embodiment is any combination of the first 7 embodiments, wherein the carrier film is translucent or transparent.

A 9^{th} embodiment is any combination of the first 8 embodiments, further comprising an additional adhesive disposed between the carrier film and the hydrocolloid layer, wherein the carrier film is in contact with the hydrocolloid layer.

A 10^{th} embodiment is any combination of the first 9 embodiments, wherein the additional adhesive is translucent or transparent.

An 11^{th} embodiment is any combination of the first 10 embodiments, further comprising a release liner.

A 12^{th} embodiment is any combination of the first 11 embodiments, wherein the base polymer comprises about 5% to about 20% by weight of the composition.

A 13^{th} embodiment is any combination of the first 12 embodiments, wherein the hydrocarbon tackifier comprise about 25% to about 40% by weight of the composition.

A 14^{th} embodiment is any combination of the first 13 embodiments, wherein the absorbent comprises about 30% to about 50% by weight of the composition.

A 15^{th} embodiment is any combination of the first 14 embodiments, wherein the plasticizer comprises up to about 20% by weight of the composition.

A 16^{th} embodiment is any combination of the first 15 embodiments, wherein the wound dressing has a refractive index from about 1.0 to about 1.7.

In another aspect, a first embodiment is a hydrocolloid adhesive composition comprising a base polymer, a tackifier, an absorbent, a plasticizer and wherein the hydrocolloid adhesive composition is substantially transparent.

A second embodiment is the first embodiment, wherein the composition has a refractive index ranging from about 1 to about 2.

A third embodiment is any combination of the first two embodiments, wherein the composition has a refractive index from about 1.0 to about 1.7.

A 4^{th} embodiment is any combination of the first 3 embodiments, wherein the composition has a total transmittance of at least about 50%.

A 5^{th} embodiment is any combination of the first 4 embodiments, wherein the total transmittance is at least about 90%.

A 6^{th} embodiment is any combination of the first 5 embodiments, wherein the base polymer comprises about 5% to about 20% by weight of the composition.

A 7^{th} embodiment is any combination of the first 6 embodiments, wherein the tackifier comprises a hydrocarbon tackifier in about 25% to about 40% by weight of the composition.

An 8^{th} embodiment is any combination of the first 7 embodiments, wherein the absorbent comprises about 30% to about 50% by weight of the composition.

A 9^{th} embodiment is any combination of the first 8 embodiments, wherein the plasticizer comprises up to about 20% by weight of the composition.

In another aspect, a first embodiment is a hydrocolloid adhesive composition comprising a base polymer, a hydrocarbon tackifier, wherein the hydrocarbon tackifier is a cycloaliphatic hydrocarbon modified with aromatic functional groups, an absorbent, and a plasticizer.

A second embodiment is the first embodiment, wherein the cycloaliphatic hydrocarbon modified with aromatic functional groups comprises about 5 wt. % to about 15 wt. % aromatic protons.

A third embodiment is any combination of the first two embodiments, wherein the cycloaliphatic hydrocarbon modified with aromatic functional groups has a softening point of from about 80° C. to about 100° C.

A 4^{th} embodiment is any combination of the first 3 embodiments, wherein the cycloaliphatic hydrocarbon modified with aromatic functional groups has a number average molecular weight of about 300 g/mol to about 600 g/mol.

A 5^{th} embodiment is any combination of the first 4 embodiments, wherein the cycloaliphatic hydrocarbon modified with aromatic functional groups has a weight average molecular weight of about 600 g/mol to about 800 g/mol.

A 6^{th} embodiment is any combination of the first 5 embodiments, wherein the cycloaliphatic hydrocarbon modified with aromatic functional groups comprises a C3 to C10 cycloaliphatic hydrocarbon resin.

A 7^{th} embodiment is any combination of the first 6 embodiments, wherein the base polymer comprises about 5% to about 20% by weight of the composition.

An 8^{th} embodiment is any combination of the first 7 embodiments, wherein the hydrocarbon tackifier comprises about 25% to about 40% by weight of the composition.

A 9^{th} embodiment is any combination of the first 8 embodiments, wherein the absorbent comprises about 30% to about 50% by weight of the composition.

A 10^{th} embodiment is any combination of the first 9 embodiments, wherein the plasticizer comprises up to about 20% by weight of the composition.

What is claimed is:

1. A hydrocolloid adhesive composition comprising:
a base polymer;
a hydrocarbon tackifier;
an absorbent; and
a plasticizer.

2. The hydrocolloid adhesive composition of claim 1, wherein the base polymer comprises about 5% to about 20% by weight of the composition.

3. The hydrocolloid adhesive composition of claim 1, wherein the hydrocarbon tackifier comprise about 25% to about 40% by weight of the composition.

4. The hydrocolloid adhesive composition of claim 1, wherein the absorbent comprises about 30% to about 50% by weight of the composition.

5. The hydrocolloid adhesive composition of claim 1, wherein the plasticizer comprises up to about 20% by weight of the composition.

6. The hydrocolloid adhesive composition of claim 1, wherein the hydrocarbon tackifier is a cycloaliphatic hydrocarbon modified with aromatic functional groups.

7. The hydrocolloid adhesive composition of claim 2, wherein the cycloaliphatic hydrocarbon modified with aromatic functional groups comprises about 5 wt. % to about 15 wt. % aromatic protons.

8. The hydrocolloid adhesive composition of claim 1, wherein the base polymer comprises a poly(styrene-olefin-styrene) polymer or a poly(styrene-isoprene-styrene) polymer.

9. The hydrocolloid adhesive composition of claim 1, wherein the absorbent is a hydrophilic polymer.

10. The hydrocolloid adhesive composition of claim 9, wherein the hydrophilic polymer is a cellulose.

11. The hydrocolloid adhesive composition of claim 1, wherein the plasticizer is selected from mineral oil, liquid isoprene rubber and combinations thereof.

12. A wound dressing comprising:
a carrier film; and
a hydrocolloid layer disposed on the carrier film, wherein the hydrocolloid layer comprises:
a base polymer;
a hydrocarbon tackifier;
an absorbent; and
a plasticizer.

13. The wound dressing according to claim 12, wherein the carrier film is selected from the group consisting of a polyurethane film, an ethylene vinyl acetate film, or a combination thereof.

14. The wound dressing of claim 12, wherein the hydrocolloid layer is discontinuous.

15. The wound dressing of claim 12, wherein the hydrocolloid layer comprises a plurality of undulating lines of the hydrocolloid adhesive composition disposed on the carrier film.

16. The wound dressing of claim 12, wherein the hydrocolloid layer comprises a plurality of dots of the hydrocolloid adhesive composition disposed on the carrier film.

17. The wound dressing of claim 12, wherein the hydrocolloid layer comprises a plurality of parallel lines of the hydrocolloid adhesive composition disposed on the carrier film.

18. A hydrocolloid adhesive composition comprising:
a base polymer;
a tackifier;
an absorbent;
a plasticizer; and
wherein the hydrocolloid adhesive composition is substantially transparent.

19. The hydrocolloid adhesive composition of claim 18, wherein the composition has a refractive index ranging from about 1 to about 2.

20. The hydrocolloid adhesive composition of claim 18, wherein the composition has a total transmittance of at least about 50%.
